(19) United States
(12) Patent Application Publication
(10) Pub. No.: US 2008/0281318 A1
(43) Pub. Date: Nov. 13, 2008

(54) Systems and methods for inductive heat treatment of body tissue

(75) Inventors: Mathieu Herbette, Mountain View, CA (US); Curtis Tom, San Mateo, CA (US)

Correspondence Address: PATENT OFFICE OF DR. CHUNG S. PARK, P. O. BOX 62312, SUNNYVALE, CA, 94088-2312, US

(73) Assignee: Tessaron Medical, Inc., Sunnyvale, CA (US)

(21) Appl. No.: 11/823,379

(22) Filed: Jun. 27, 2007

Related U.S. Application Data
(63) Continuation-in-part of application No. 11/801,453, filed on May 9, 2007.

(51) Int. Cl. A61B 18/18

(52) U.S. Cl. 606/41

(57) ABSTRACT

Apparatus and methods for treating body tissue by heat energy. The apparatus includes: a coil for transmitting an alternating electrical current therethrough to generate an alternating electromagnetic field that is capable of exciting material positioned in the body, the material being operative to inductively generate the heat energy in response to the electromagnetic field; means for measuring an amplitude of the current; and means for mapping the amplitude into a temperature of the magnetic material.

### CROSS REFERENCE TO RELATED APPLICATIONS

This application is a continuation-in-part of U.S. patent application Ser. No. 11/801,453, entitled “Systems and Methods for Treating Body Tissue” by Tom et al., filed on May 9, 2007, and incorporated by reference in its entirety.

### BACKGROUND

The present disclosure generally relates to medical methods and apparatus, more particularly, to treating various types of body tissue by RF inductive heating.

Human and/or animal can suffer from various types of tissue-related illnesses, such as breast cancer and tumors. One of the approaches to treat the illness is thermotherapy. Thermotherapy, such as hyperthermia treatment, in which miniscule particles including ferromagnetic material are injected into the target tissue and then the malignant cells are destroyed by subsequent overheating by external alternating magnetic fields, enables a targeted overheating of only the region of the target tissue. The particles may be coated with a molecular layer of enzyme that has an affinity to the malignant cells.

In the hyperthermia treatment, diseased tissue may be treated by elevating the temperature of its individual cells to a lethal level. For instance, temperatures in a range about 40° C. to about 45° C. can cause irreversible damage to diseased cells, while healthy cells may survive exposure to temperature up to around 46.5° C. As such, a precise control of the temperature is needed for safe and effective hyperthermia treatments.

A known technique to measure the temperature of the target tissue during treatment may be inserting a temperature probe into the tissue in advance and reading the signal from the probe during treatment, as disclosed in U.S. Patent Application Publication No. 2005/0159780 A1. This technique has difficulties in treating certain types of tissue. For instance, brain tumor may require drilling a hole in the skull to push the probe into the tissue. For another instance, the invasive breast cancer cells break free of where they originate, invading the surrounding tissues that support the ducts and lobules of the breast. In this case, a temperature probe may be used to monitor the temperature of a limited area at the best. Depending on the size and distribution of the diseased lobules, a treatment may require multiple insertions of a probe into different locations of the breast, which may be time consuming and unpleasant to the patient.

Another difficulty in hyperthermia treatments may be locating the injected particles such that the external magnetic field is effectively coupled to the particles during operation. A known technique to locate the injected particles may be the real-time fluoroscopy using X-ray that can damage healthy cells if overexposed inadvertently. Even if the location of injected particles is known, existing systems may be able to position the external magnetic fields relative to the particles. As such, there is a strong need for systems and methods to precisely position the external magnetic field relative to the injected particles and monitor the temperature of the particles during treatment.

### SUMMARY OF THE DISCLOSURE

In one embodiment, an apparatus for treating tissue of a body by heat energy includes: a coil for transmitting an alternating electrical current therethrough to generate an alternating electromagnetic field that is capable of exciting material positioned in the body, the material being operative to inductively generate the heat energy in response to the electromagnetic field; means for measuring a quantity associated with the current; and means for mapping the amplitude into a temperature of the magnetic material.

In another embodiment, a method for treating tissue of a body by heat energy includes steps of: disposing material into the body, the material being operative to inductively generate the heat energy in response to an electromagnetic field external to the body; transmitting an alternating current through a coil to generate the electromagnetic field thereby causing the material to generate the heat energy; measuring a quantity associated with the current; mapping the measured quantity into a temperature of the material; and displaying the temperature.

### BRIEF DESCRIPTION OF THE DRAWINGS

FIG.1 shows a schematic diagram of a system for treating target tissue in accordance with one embodiment of the present invention;

FIG. 2 shows a schematic perspective view of a paddle in FIG. 1;

FIG. 3 shows a schematic side view of the paddle in FIG. 2;

FIG. 4 shows a schematic front view of a coil unit included in the paddle in FIG. 2;

FIG. 5 shows a schematic cross sectional diagram of the coil unit in FIG. 4, taken along the line V-V;

FIG. 6 shows a schematic perspective view of a paddle in accordance with another embodiment of the present invention; and

FIG. 7 shows a schematic side view of the paddle in FIG. 6.

### DETAILED DESCRIPTION OF THE PREFERRED EMBODIMENTS

The following detailed description is of the best currently contemplated modes of carrying out the invention. The description is not to be taken in a limiting sense, but is made merely for the purpose of illustrating the general principles of the invention because the scope of the invention is best defined by the appended claims.

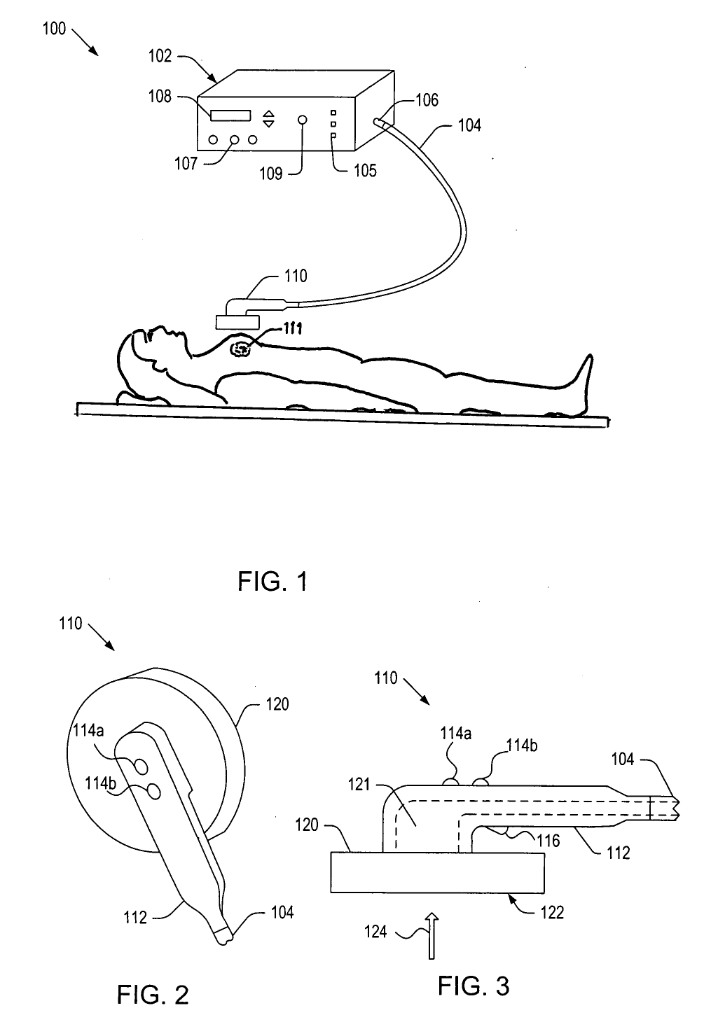

Referring now to FIG. 1, FIG.1 shows a schematic diagram of a system 100 for treating target tissue in accordance with one embodiment of the present invention. For the purpose of illustration, a breast cancer is shown as exemplary target tissue 111. However, it should be apparent to those of ordinary skill that the system 100 can be used to treat other suitable diseased and/or malignant tissue as well as blood vessels, such as varicose veins to be destroyed. As depicted, system includes a controller 102, a paddle 110, and an umbilical cord 104 for connecting the paddle 110 to the controller 102. In an exemplary embodiment, the umbilical cord 104 may be connected to the controller 102 via a detachable connector 106 that permits various types of the paddle to be detachably connected to the controller 102.

As will be detailed, the electromagnetic field generated by the paddle 110 may excite particles delivered into the patient's body. In some applications, the electromagnetic flux generated by the paddle 110 may not penetrate deep into the patient's body to reach the target tissue. In another exemplary embodiment, multiple paddles may be coupled to the controller 102 via multiple umbilical cords 104. For instance, two paddles arranged in opposite with respect to the patient's body may be activated simultaneously, forming a Helmholtz configuration to cause the electromagnetic flux generated by the coil to penetrate deep into the body. In yet another exemplary embodiment, the controller 102 may be used to operate a coil dimensioned to surround a portion of the patient's body so that the electromagnetic flux generated by the coil may penetrate deep into the body.

The controller 102 includes indicators 105, 107, a display panel 108, on/off switch 109, or the like. The indicators 105 may be LED light indicators and indicate the operational status, such as power on, fault conditions, activation of magnetic filed, or the like. The indicators 107, preferably LED light indicators, may indicate operational status, such as cooling fluid in/out, power to the paddle, or the like. The display 108 may display various quantities, such as the temperature of the particles during treatment, time lapsed in each treatment cycle, or the like. It is noted that the number and size of indicators, switch, and display panel on the controller 102 may be varied without deviating from the scope and spirit of the present teachings.

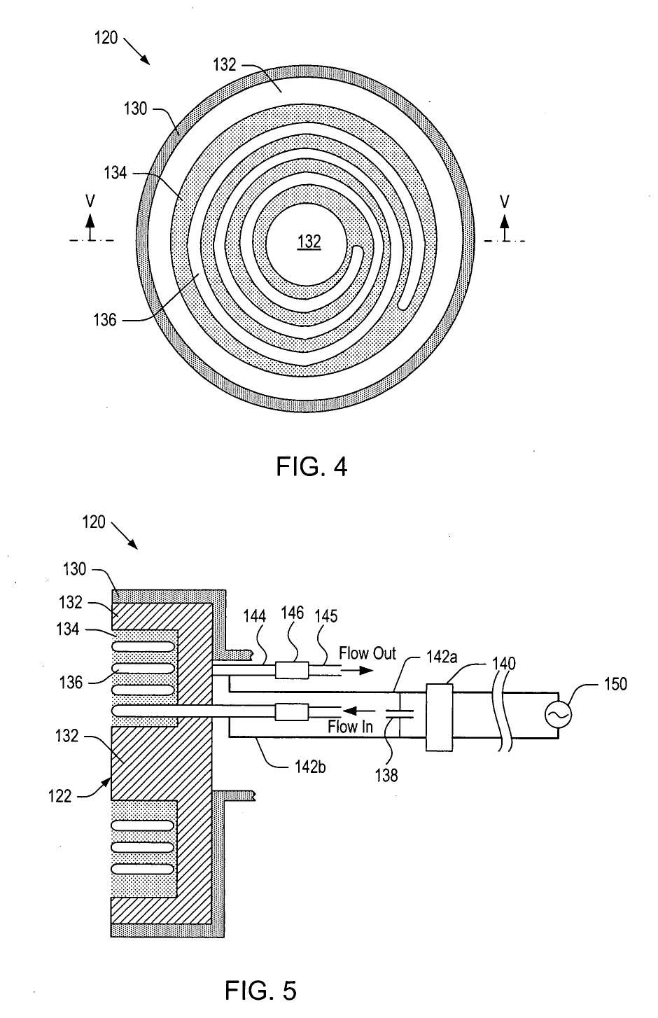

The controller 102 includes a microprocessor based subsystem that converts main (AC) power to a high frequency alternating current source. The alternating current, which is preferably in the radio frequency (RF) range, is applied to the paddle 110, more specifically, electrical coil, to generate alternating electromagnetic field. As will be detained in conjunction with FIGS. 4-5, the coil may be made from a conducting tube through which cooling fluid passes. The controller 102 may include a pump for circulating the cooling fluid through the coil and a heat exchanger for dissipating the heat energy from the cooling fluid. Some of the indicators 107 may be used to indicate the flow through the coil. The controller 102 may include an automatic feedback control subsystem to monitor the cooling fluid temperature and regulate the flow rate to regulate the fluid temperature.

The controller 102 may include a user programmable timer that allows a user to set time interval for treatment such that the system 100 will de-energize the coil after a predetermined amount of elapsed time. The controller 102 may also include a control button that allows a user to set the time interval manually.

The controller 102 may also control the amount of energy (in the form of heat) delivered to the target tissue 111. In one exemplary embodiment, the controller 102 may measure the amount of time the target tissue 111 is at the target temperature. By use of a closed loop control subsystem included in the controller 102, the heat loss due to conduction via blood vessels during treatment can be taken into account in determining the amount of time. The liver, for example, is highly vascular and would present a large thermal heat sink. Operating in a purely timed mode may result in under treatment of such target tissue.

FIGS. 2 and 3 show schematic perspective and side views of the paddle 110 in FIG. 1. As illustrated, the paddle 110 includes a handle 112 and a coil unit 120 secured to the handle 112. The handle 112 may include a control switch 116 to activate a coil in the coil unit 120 and two indicators 114a, 114b, which are preferably LED light indicators and operative to indicate the operational status of the system 100, such as the controller power and activation of coil in the paddle 110. The handle 112 is preferably, but not limited to, formed of hollow plastic defining a cavity or empty space 121 and configured to provide enhanced grip and ergonomic comfort for the user. One end of the umbilical cord 104 is connected to the handle 112 such that several electrical wires (not shown in FIG. 2) in the umbilical cord 104 can extend into the cavity 121. The electrical wires are connected to the indicators 114a, 114b and controller 102. In one exemplary embodiment, to activate the coil in the coil unit 120, the user may operate the switch 116 coupled to the controller 102 via a pair of electrical wires extending through the cavity 121 to the controller 102. In another exemplary embodiment, a foot operated switch (not shown in FIG. 2) may be coupled to the controller 102 so that the user can remotely operate the coil unit 120 by operating the switch. In yet another exemplary embodiment, other types of switches, such as pneumatic or optical switch, may be used to operate the coil unit 120.

A pair of tubes for providing cooling fluid to the coil unit 120 also extend from the controller 102 through the umbilical cord 104 and cavity 121. It is noted that the handle 112 may include other indicators and display panels. For instance, the temperature of the particles measured by the system 100 can be displayed on one display panel. For another instance, the time elapsed in each treatment cycle can be displayed on another display panel. For yet another instance, an LED indicator for flow in the coil unit 120 may be mounted on the handle 112.

The coil unit 120 generates alternating electromagnetic field that excites particles delivered into the target tissue 111. Systems and methods for delivering the particles into various target tissues are disclose in U.S. patent application Ser. No.______ , entitled “Systems and Methods for Delivering Particles Into Patient Body,” filed on Jun. 27, 2007, which is herein incorporated by reference in its entirety.

FIG. 4 shows a schematic front view of the coil unit 120 seen along the direction 124 (FIG. 3). FIG. 5 shows a schematic cross sectional diagram of the coil unit 120, taken along the line V-V (FIG. 4). As depicted, the coil unit 120 has a generally cylindrical shape and includes: an outer housing 130 formed of electrically insulating material; a flux concentrator 132 secured to the inner surface of the housing and having a generally cylindrical shape with a U-shaped channel formed in the front surface portion thereof; and inductor coil 136 disposed in the channel and secured to the flux concentrator by electrically insulating adhesive or glue 134 so that the coil 136 is electrically insulated from the flux concentrator 132.

The outer housing 130 is securely connected to the handle 112. In one exemplary embodiment, the housing 130 and handle 112 are formed in one integral body. The flux concentrator 132 may be formed of material with a high permeability, wherein the material may include semi-conducting or non-conducting material, such as ferrite. The material may also include conducting material, such as nickel alloy. The flux concentrator 132 may block the electromagnetic flux propagating rearward, perhaps toward the user, and redirect the blocked magnetic flux toward the front surface 122 of the coil unit 120.

The ideal flux concentrator would not heat up if it were 100% efficient. However, the flux concentrator 132 may get warm as it is somewhat lossy, i.e., a portion of the flux is converted into heat energy by the concentrator. To minimize heat build up in the flux concentrator 132, a thermally conductive (but not electrically conductive) glue 134, such as heat epoxy, can be use to transport the heat from the concentrator 132 to the liquid cooled coil 136.

The coil 136 is formed of a metal tube, such as copper, and coupled to a power source 150 that may be included in the controller 102 via a pair of electrical wires 142a, 142b. The power source 150, which is preferably an RF power source, may be activated by the switch 116 on the handle 112 and/or a switch on the controller 102. Two end portions 144 of the coil 136 extend through the flux concentrator 132 to couplers 146. Each coupler 146 couples one end of the coil to a flexible tube 145 connected to the controller 102 for cooling fluid communication. The flexible tube 145, positioned in the umbilical cord 104, may be formed of, but not limited to, polymer and operative to carry cooling fluid from the controller 102 to the coil 136.

Typically, the alternating current applied to the coil 136 propagates along the surface of the coil, more specifically, within a skin depth from the surface. Thus, to increase the intensity of electromagnetic flux emitted by the coil unit 102, the surface area or turn density (number of turn/coil diameter) of the coil 136 needs to be increased. The coil 136 has a generally flattened tubular shape that permits a larger number of coil turns for a given channel in the flux concentrator 132, increasing the surface area of the coil 136 thereby to enhance the flux intensity. In one exemplary embodiment, the coil 136 may be formed of copper tube and the surface of the coil 136 may be plated with a highly conductive material, such as silver, to enhance flux intensity. As the coil 136 may be operated at high frequencies, most of the electrical current may flow along the surface of the copper coil, i.e., the current may flow through the highly conductive material.

The coil unit 120 may include a capacitor 138 forming an LC tank circuit with the coil 136. In one exemplary embodiment, the capacitor 138 may be located in the handle 112 in close proximity to the coil 136. A matching network 140, which may be located in either coil unit 120 or controller 102, can be coupled to the electrical wires 142a, 142b to match the output impedance of the controller 102 to that of the coil 136.

The paddle 110 is designed to activate particles that are within a preset distance, for instance 0 to 2 centimeters, from the front surface 122 of the paddle 110. Generally speaking, the particles are better coupled with the external electromagnetic field when located inside the projection area of the front surface 122 of the flux concentrator 132. Each paddle 110 may be assigned a unique ID associated with information of operational characteristics, such as the preset distance, duty cycle, and resonant frequency, as well as effective projection area. The information may also include the expected target mass, i.e., the expected amount of particle mass to be heated by the paddle 110.

The particles delivered to the target tissue 111 may be formed of material that can generate heat energy in response to the electromagnetic field generated by the coil 120. The material includes, but is not limited to, metal, plastic, polymer, ceramic, or alloys thereof. Typically, the permeability of material for the particles decreases as its temperature increases up to Curie temperature at which the material becomes paramagnetic. During operation, the coil 136 can be electromagnetically coupled with the particles, causing the resonant frequency of the LC tank circuit (coil 136 and capacitor 138) to shift from the unloaded frequency. Hereinafter, the term unloaded (or, equivalently, reference) refers to a state where the coil is located remotely from the particles. As the temperature of the particles changes, this shift may also change and, as a consequence, the amplitude of the current or voltage in the coil 136 may also change. Hereinafter, the term amplitude refers to peak-to-peak or RMS value of the alternating current or voltage. In one exemplary embodiment, the controller 102 may include a closed-loop control subsystem for tuning the operational frequency of the power source 150 to the resonant frequency of the LC tank circuit thereby to optimize the operational efficiency of the coil 136.

As the amplitude of the current in the coil 136 changes, the intensity of the electromagnetic field may also change. In one exemplary embodiment, the closed-loop control subsystem may continuously monitor the amplitude of the current and/or voltage in the coil 136 and vary one or more operating parameters, such as the voltage, frequency, and duty cycle of the power source 150, etc., to maintain optimum operation of the coil 136. In another embodiment, the change in amplitude of the current or voltage in the coil 136 can be monitored and mapped into the change in the particle temperature, i.e., the measured amplitude or voltage of the current can be mapped into the particle temperature. For given dose of particle delivered to the target tissue, the relationship between the current or voltage in the coil 136 and the particle temperature can be obtained. Then, based on the relationship, the measured amplitude or voltage of the current in the coil 136 can be used to read the temperature of the particles. The particle temperature may be displayed on a display window mounted on the controller 102 and/or handle 112.

In one exemplary embodiment, the current in the coil 136 can be measured by a current sensing transformer in line with the circuit feeding current into the coil 136. In another exemplary embodiment, the current in the coil 136 can be measured by a coil or loop disposed in close proximity to the coil 136 that can pick up the alternating magnetic field generated by the coil 136. The controller 102 may include a suitable circuit and a microprocessor that may map the measured current into the particle temperature.

Each paddle 110 (or coil) may have a unique ID that allows the controller 102 to set the optimum frequency and duty cycle for operation of the coil 136. To aid the user in positioning the paddle relative to the patient body so as to place the particles within the optimum working range of the alternating electromagnetic field generated by the paddle 110, the controller 102 may be switched to a “guidance” mode. In the guidance mode, the controller 102 may activate the paddle 110 at the unloaded resonant frequency associated with the unique ID. Then, as the user moves the paddle 110 around the patient body, the controller 102 may send an audio and/or visual signal indicating the shift or deviation of the amplitude of the current or voltage in the coil 136 from a reference value, such as the amplitude at unloaded state. Typically, the greater the deviation is, the closer the paddle is located to the particles. In one embodiment, the controller 102 (or the handle 112) may increase the beeping sound frequency as the shift increases, aiding the user in finding the optimum location of the paddle 110 relative to the particles. In another embodiment, the controller 102 (or the handle 112) may increase the light intensity of an LED as the shift increases.

It is noted that the measured deviation in the guidance mode can be used as a safety feature. If the paddle 110 is inadvertently placed on a large ferromagnetic mass, such as an examination table, the controller 102 may sense the larger than expected mass and will not activate the system 100.

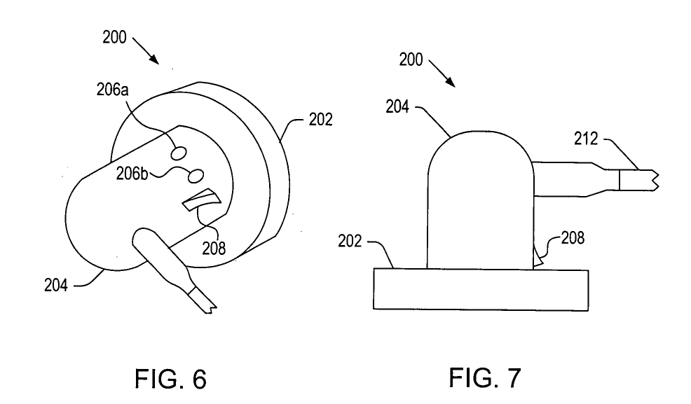

FIGS. 6 and 7 show schematic perspective and side views of a paddle in accordance with another embodiment of the present invention. As depicted, the paddle 200 includes a handle 204 and a coil unit 202 secured to the handle. The handle includes a control switch 208 to activate a coil in the coil unit 202 and two indicators 206a, 206, which are preferably LED light indicators. The handle 204 is connected to an umbilical cord 212 and have similar structure as the handle 112 in FIGS. 2-5. As the paddle may have similar structural and operational mechanisms as the paddle 110 (FIG. 2-3), detailed description of the paddle 200 is not repeated for brevity.

It is noted that the system 100 can be used with other types of heat generating masses in place of the particles. For instance, various types of catheters disclosed in the parent U.S. application, Ser. No. 11/801,453, entitled “Systems and Methods for Treating Body Tissue,” and filed on May 9, 2007, can be inserted in the patient body for the similar hyperthermia treatment. Detailed description of how to operate the system 100 in conjunction with the catheters is not repeated for brevity since the interaction of the paddle 110 with the heat generating portions of the catheters disclosed in the parent application may be similar to the interaction between the paddle and particles.

It should be understood, of course, that the foregoing relates to exemplary embodiments of the invention and that modifications may be made without departing from the spirit and scope of the invention as set forth in the following claims.

What is claimed is:

1. An apparatus for treating tissue of a body by heat energy, comprising:
a coil for transmitting an alternating electrical current therethrough to generate an alternating electromagnetic field that is capable of exciting material positioned in the body, the material being operative to inductively generate the heat energy in response to the electromagnetic field;
means for measuring a quantity associate with the current; and
means for mapping the amplitude into a temperature of the magnetic material.

2. An apparatus as recited in claim 1, wherein the quantity is an amplitude of the current.

3. An apparatus as recited in claim 1, wherein the quantity is an amplitude of a voltage applied to the coil.

4. An apparatus as recited in claim 1, further comprising:
a display for displaying the temperature.

5. An apparatus as recited in claim 1, wherein the current alternates at an RF frequency.

6. An apparatus as recited in claim 1, wherein the coil is formed of a tube.

7. An apparatus as recited in claim 1, further comprising:
a flux concentrator having a channel formed in a surface portion thereof, the coil being disposed in the channel so that electromagnetic flux generated by the coil is emitted from the surface portion.

8. An apparatus as recited in claim 1, wherein the means for measuring a quantity includes a measuring coil disposed in proximity to the coil.

9. An apparatus as recited in claim 1, wherein the means for measuring a quantity includes a loop disposed in proximity to the coil.

10. An apparatus as recited in claim 1, further comprising:
means for sending a signal indicating a deviation of the quantity from a reference value.

11. An apparatus as recited in claim 10, wherein the signal includes a beeping sound having a frequency commensurate with the deviation.

12. An apparatus as recited in claim 10, wherein the signal includes a light signal having an intensity commensurate with the deviation.

13. An apparatus as recited in claim 1, wherein the material includes at least one particle.

14. An apparatus as recited in claim 1, wherein the material forms a portion of a catheter.

15. A method for treating tissue of a body by heat energy, comprising:
delivering material into the body, the material being operative to inductively generate the heat energy in response to an electromagnetic field external to the body;
transmitting an alternating current through a coil to generate the electromagnetic field thereby causing the material to generate the heat energy;
measuring a quantity associated with the current;
mapping the measured quantity into a temperature of the material; and
displaying the temperature.

16. A method as recited in claim 15, wherein the coil is formed of a tube and wherein the step of transmitting an alternating current includes causing cooling fluid to pass through the coil.

17. A method as recited in claim 15, wherein the coil is disposed in a channel formed in a surface portion of a flux concentrator, further comprising:
directing electromagnetic flux generated by the coil so that the flux is emitted from the surface portion.

18. A method as recited in claim 15, wherein the step for measuring a quantity is performed by use of a measuring coil disposed in proximity to the coil.

19. A method as recited in claim 15, wherein the step for measuring a quantity is performed by use of a loop disposed in proximity to the coil.

20. A method as recited in claim 15, further comprising:
sending a signal indicating a deviation of the measured quantity from a reference value.

21. A method as recited in claim 20, wherein the step of sending a signal includes sending beeping sounds at a frequency commensurate with the deviation.

22. A method as recited in claim 20, wherein the step of sending a signal includes sending a light signal having an intensity commensurate with the deviation.

23. A method as recited in claim 15, wherein the step of measuring a quantity includes measuring an amplitude of the current.

24. A method as recited in claim 15, wherein the step of measuring a quantity includes measuring an amplitude of a voltage applied to the coil.
